(19) United States
(12) Patent Application Publication
(10) Pub. No.: US 2023/0000799 A1
(43) Pub. Date: Jan. 5, 2023

(54) EFFECTS OF MESCALINE AND OF MESCALINE ANALOGS (SCALINES) TO ASSIST PSYCHOTHERAPY

(71) Applicant: Universitätsspital Basel, Basel (CH)

(72) Inventor: Matthias Emanuel LIECHTI, Oberwil (CH)

(73) Assignee: Universitätsspital Basel, Basel (CH)

(21) Appl. No.: 17/883,502

(22) Filed: Aug. 8, 2022

Related U.S. Application Data
(63) Continuation of application No. 17/675,894, filed on Feb. 18, 2022.
(60) Provisional application No. 63/153,318, filed on Feb. 24, 2021.

(51) Int. Cl. A61K 31/137; A61P 25/00

(52) U.S. Cl. CPC A61K 31/137; A61P 25/00

(57) ABSTRACT

A method of inducing psychedelic states in an individual, by administering mescaline, a salt thereof, analogs thereof, or derivatives thereof to the individual, and inducing a psychedelic state in the individual. A method of therapy, by administering an intermediate “good effect dose” of mescaline, salt of mescaline, analogs thereof, or derivatives thereof to an individual, and inducing positive acute drug effects that are known to be associated with more positive long-term responses in psychiatric patients. A method of therapy, by administering an “ego-dissolution” dose of mescaline, a salt of mescaline, analogs thereof, or derivatives thereof to an individual, and providing the experience of ego-dissolution.

### GRANT INFORMATION

The research has in part been supported by a grant from the Swiss Science National Foundation (nr. 320036_185111) to Matthias Liechti.

### BACKGROUND OF THE INVENTION

### 1. Technical Field

The present invention relates to the use of mescaline and mescaline analogs or derivatives to induce a psychedelic state and assisting psychotherapy and treating medical condition.

### 2. Background Art

Hallucinogens or psychedelics are substances capable of inducing exceptional subjective effects such as a dream-like alteration of consciousness, pronounced affective changes, enhanced introspective abilities, visual imagery, pseudo-hallucinations, synesthesia, mystical-type experiences, and experiences of ego dissolution (1-3).

Efficacy of psychedelics for the treatment of medical conditions has been shown in clinical trials using lysergic acid diethylamide (LSD) and in patients with addiction (4), in patients with anxiety associated with life-threatening illness (5, 6), and using psilocybin in patients with major depression (7-11), anxiety disorder or anxiety associated with terminal illness (9, 10, 12), and in different forms of addiction (13-18). There is also evidence that the psychedelic brew Ayahuasca, which contains the active psychedelic substance N,N-dimethyltryptamine (DMT) (19) may alleviate depression (20-22). In contrast, there are no therapeutic trials or elaborated scientific concepts regarding the use of the psychedelic substance mescaline (3,4,5-trimethoxyphenethylamine) in the treatment of medical conditions.

Psychedelics such as psilocybin and LSD can be used to assist psychotherapy for many indications including anxiety, depression, addiction, personality disorder, and others, and can also be used to treat other medical disorders such as cluster headache and migraine and others. Although no psychedelic is currently licensed for medical use, psilocybin and LSD are used already experimentally within clinical trials and special therapeutic (compassionate use) programs (4, 5, 9, 10, 12, 17, 18, 23, 24). There is no comparable therapeutic use of mescaline.

Additionally, existing psychedelic treatments such as LSD, psilocybin, and DMT may not be suitable to be used in all patients suited for psychedelic-assisted therapy. The availability of several substances including novel ones with different properties is important and the present lack thereof is a therapeutic problem which will further increase with more patients needing psychedelic-assisted therapy and an increase in demand for such treatment once the efficacy of first treatments (psilocybin and LSD) will be documented in large clinical studies. For example, some patients may react with strong adverse responses to existing therapies such as psilocybin presenting with untoward effects including headaches, nausea/vomiting, anxiety, cardiovascular stimulation, or marked dysphoria. In such patients, mescaline is useful as alterative treatment to the psychedelic that produced adverse responses. In some patients, mescaline can also be useful because another experience than made with psilocybin or LSD is necessary or because a patient is not suited for therapy with these existing approaches a priori. Thus, mescaline and its derivatives can serve as alternative treatment options with characteristics sufficiently similar to other psychedelics to be therapeutic but also sufficiently different to provide added benefits or avoid negative effects of other psychedelics.

Mescaline was described early as being useful in psychiatric research to help with the discovery of the ethology of psychoses (25) and as “means to better understanding madness or the human mind” (26). A use as therapeutic tool was also mentioned (27). Several older case reports or case series were reported.

Mescaline was studied in schizophrenic patients and found to “markedly aggravate their mental symptomatology” and to induce a “disorganization of the psychic integration” in both schizophrenics and normal subjects (28). A dose of “0.5 grams of mescaline was noted to usually suffice to evoke hallucinatory an affective responses in most subjects” (27). Turns and Denber described the use of mescaline in two patients (29). One patient was diagnosed with schizophrenia, one with severe depression. Both were considered resistant to standard pharmacological treatment. Mescaline was administered weekly in doses of 500 mg orally in one and intravenously in the other patient. The sessions were terminated with administration of chlorpromazine intravenously. The patient with schizophrenia received 8 mescaline treatments and the patient with depression received 12 treatments. The patient with depression reportedly improved. Both patients reportedly benefited from effects of mescaline “to accelerate/promote psychotherapy” (29). Smith reported the use of mescaline in the treatment of alcoholism in a case series (30). Doses of 0.5 grams were used in seven patients in conjunction with psychotherapy. There was no control group or statistical analysis of the data. Favorable responses were reported but it was not possible to define the effect of mescaline separate from the psychotherapy (30). Use of mescaline “drug-induced states” as an aid in psychotherapy has also described in a case report by the psychiatrist Walter Frederking (31). A case of successful treatment with mescaline and psychotherapy for erectile dysfunction and a marriage improvement were described. The psychiatrist noted that mescaline (300-500 mg sulfate intramuscularly) was more intense and overpowering than LSD tartrate (30-60 μg orally) and more difficult to dose and that LSD had a broader spectrum in its physical effects (31). No systematic use descriptions of mescaline for specific diseases or indications have been reported and the dosing definitions are lacking.

Buchanan reported on the religious use in its publication on the “Meskalinrausch” in 1929 (32). He noted that “the sacred ritual of the eating of peyote occupies a period of about twelve to fourteen hours.” The Indians regard mescal as a panacea in medicine, as a source of inspiration and a key which opens to them the glories of another world” (32). In 1896, Heffter extracted mescaline from mescal. Mescaline was reported to have effects on the cardiovascular system including “slowing of the heart rate and a rise in systolic pressure”. However, respiratory and cardiac depressing effects were also noted (32) and solid data on the cardiovascular effect profile of defined doses have been lacking and were generated within this invention. Early reports on the acute effects of mescaline were compiled and published by (32). For example, Prentiss and Morgan (1885) cited in (32) reported that “I could see all sorts of design in brilliant and ever-changing colors.” . . . “My mind was perfectly clear and active” . . . and “I truly thought that I had experienced great pleasure upon many previous occasions, but the experience of this was one quite unique in this regard in the history of a lifetime” (32). Mitchell, cited in (32), reported “I had a certain sense of the things about me as having a more positive existence than usual. It is not easy to define what I meant, and at the time I searched my vocabulary for phrases or word which should fitly state my feeling. It was in vain.” It is noted that “subjects forget themselves and place and time become dominated by a feeling of absolute timelessness. There is a feeling of monumental existence, a sphinxlike experience” (32). These citations illustrate that mescaline has previously been reported to induce positive and unique experiences in humans that are difficult to describe with words.

Mescaline was the first hallucinogen used in psychiatric research, however, not as treatment but as a tool to mimic and study psychosis (33-35). These are also early scientific descriptions of the acute alterations of mind induced by psychedelics. There have been several descriptions of the mescaline effects but (25, 33, 36-39) but only few more modern studies (40, 41) on the effects of mescaline in humans. There are no contemporary studies using modern scientific psychometric methods.

In past studies in healthy subjects, effects of mescaline were compared with those of placebo and the 3,4-methylendioxymethamphetamine (MDMA)-like substance MDEA in the 1990ies (40, 41), but a modern and methodologically valid comparison with other psychedelics like LSD and psilocybin that are currently being used in patients is lacking.

There was an older study that directly compared mescaline (5 mg/kg), psilocybin (0.15 mg/kg), and LSD (0.01 mg/kg) in 18 subjects (36). The substances generally produced similar effects and were considered to have been equivalent regarding their overall acute effects. However, at the dose used, mescaline tended to produce more pronounced effects than psilocybin or LSD including a higher proportion of subjects with perceptual changes and nausea as well as other adverse effects. Detailed data is not available.

Another study compared mescaline (5 mg/kg), psilocybin (0.225 mg/kg) and LSD 0.015 mg/kg) and even a combination of all three drugs using one-third doses of each in 24 healthy male subjects (42). Mood effects were reported to be alike for all four treatments but, surprisingly, no perceptual or psychic effects or typical psychedelic-like effects (synesthesia, disturbed perception) were evaluated and reported (42) possibly because of the use of inappropriate methods including non-sensitive psychometric instruments.

Another old study found that subjects could not distinguish between the substances (37).

An experimenter compared in a self-study oral LSD (100 μg) with mescaline (350 mg subcutaneously) with a focus on drawing pictures of faces. LSD was reported to produce symptoms of psychopathology of the hebephrenic type while mescaline was reported to produce more catatonic type effects and more dysphoria than LSD (43).

There are no previously well-tested or well-defined medico-therapeutic uses of mescaline. One study administered mescaline to schizophrenic patients and found that mental symptoms were markedly aggravated (28). Visual hallucinations were noted to be less frequent after LSD than after mescaline indicating differences between the two (28). However, this was not documented using valid psychometric instruments.

Another preliminary study used a double-blind administration of mescaline 500 mg, psilocybin 10 mg and LSD 70 μg. Mescaline produced the most alterations in all examinations but no statistical evaluations were performed (44). The dose of mescaline was high compared to the low dose of psilocybin and thus this study did not compare equivalent doses.

An early study compared the effects of mescaline, psilocybin and LSD administered intramuscularly in humans (37). The study found similarities regarding subjective and autonomic effects and reported differences in the time courses of the three substances. Unlike contemporary research and the present invention that use oral administration, intramuscular administration of the substance was used.

In a study that could be considered more modern, Herme administered mescaline at a dose of 500 mg or mescaline-sulfate to 12 healthy male subjects. The study also used a former and already validated version of the present 5D-ASC scale called the APZ (altered states of consciousness) scale to assess the acute effects of mescaline (40). Due to differences in molecular weights, the dose of 500 mg mescaline sulfate used by Herme (40) would correspond to a dose of approximately 406 mg mescaline hydrochloride used to generate data for the present invention. Mescaline induced a state of altered consciousness with increased ratings of oceanic boundlessness (OB or OSE), anxious ego-dissolution (AEG or AIA), and visionary restructuralization (VR or VUS) in the APZ scale. An indirect comparison with the effects of the MDMA-like empathogen MDE in 14 healthy subjects showed greater average APZ scores after mescaline compared with MDE (OSE: 6.2 vs. 3.9; AIA 7.1 vs. 2.6; VUS 7.4 vs. 1.6) including mainly relatively greater anxious ego-dissolution and perceptual alterations for mescaline compared with MDE. This pattern is very broadly similar to the comparison of the psychedelic LSD with the MDMA (OB 43 vs. 9.3; AED 26 vs. 1.3; VR 50 vs. 4.1) (45) and confirming stronger and greater perceptual effects of serotonergic psychedelics compared with empatogenic MDMA-like substances also used in substance-assisted therapy (23). Importantly, the study by Hermle used only one dose of mescaline without a direct comparison with other psychedelics and focused on imaging data (40) and not on the acute subjective and cardiovascular adverse effects relevant within the present invention.

There is no modern data on the clinical pharmacology of mescaline. An early study used C14-labeled mescaline to study the metabolism of mescaline in twelve healthy male subjects using a total dose of mescaline hydrochloride of 500 mg orally (46). Blood, urine and spinal fluid was collected repeatedly after mescaline administration. The half-life of the radioactivity ingested as mescaline was approximately six hours and 87% of the radioactivity administered was excreted within 24 hours and 92% within 48 h. Mescaline was mainly excreted in urine as unchanged mescaline (55-60%). The main metabolite was 3,4,5-trimethoxyphenylacetic acid (TMPA, 27-30%). Minor metabolites were N-acetyl-(3,4,dimethoxy-5-hydroxyphenylethalymine (5%), another 0-demethylated phenylacetic acid (HMPA, 1%) and N-acetyl-mescaline (NAM, 0.1%) and others (10%). The metabolites TMPA and NAM were also produced and administered to humans and were found not to have psychoactive or cardiostimulant properties (46). Taken together, mescaline was eliminated mainly unchanged or as inactive TMPA. Subjective effects appeared within 30 min, peaked at 4 hours and lasted 12-14 hours. There was a delay in the peak effect of the subjective response compared with the plasma peak by about 2 hours (46). A more systematic test of the duration of action in comparison with other substances with a similar action is lacking.

Phase 1 studies on mescaline with a focus on maximal tolerated dose finding and safety are lacking. Acute overdoses produce mainly a sympathomimetic toxidrome (47). A retrospective review of the California poison center database search for the years 1997-2008 found 31 exposures to peyote plants (29 cases) or mescaline (only two cases). Commonly reported effects were hallucinations, tachycardia, and agitation (48). Clinical effects were usually mild or moderate, and life-threatening toxicity was not reported in this case series (48). Treatment included sedatives or was supportive only. Similar to other psychedelics, mescaline has no abuse-related rewarding effects in rodent tests (49). There was no evidence of psychological or cognitive deficits among Native Americans using peyote regularly in a religious setting (50).

Interest in mescaline is ongoing with a focus on preclinical studies (51-54) but no studies in humans.

Although mescaline is long-known psychedelic with a history of spiritual use, information on its effects in human is scarce and old, and there is no state of the art modern scientific data on the effects of mescaline in humans or a systematic evaluation of its potential use as a therapeutic.

Acute psychedelic states relevant for therapeutic use and induced by psilocybin and LSD have been very well-characterized in modern research studies (1, 3, 7, 9, 10, 45, 55-59). However, no such data has been available on mescaline (51).

Psychedelic substances produce their characteristic acute effects in humans via activation of the serotonin 5-HT_{2A} receptor as specifically shown in clinical studies for psilocybin and LSD (3, 60, 61). All serotonergic psychedelics including LSD, psilocybin, DMT, and mescaline are agonists at the 5-HT_{2A} receptor (62) and may therefore produce overall largely similar effects. However, such confirming modern studies that directly compare acute effect profiles of different substances are lacking.

Positive acute subjective psychedelic experiences after administration of psilocybin are correlated with its long-term therapeutic benefits in patients with depression or addiction (7, 9, 15). This means that the acute effects of a serotonergic psychedelic in humans can be used to predict, at least in part, the therapeutic outcome in patients. Acute effects that may contribute to positive long-term effects of psychedelics including mescaline are effects that are thought to enhance the therapeutic relationship including increased openness, trust, feelings of connectedness or emulsion with persons, insight in psychological problems and stimulation of neuroregenerative processes as described in detail elsewhere (63).

There remains a need for studies showing that mescaline can produce acute subjective effects in an individual that are sufficiently similar to those of therapeutically used psychedelics and predict therapeutic use of mescaline and mescaline derivatives as well as a need for effective psychedelic treatments. The acute effects of mescaline have not been validly described and shown to be similar to psychedelics with therapeutic use. Additionally, there is a need for defining characteristics of mescaline different from already therapeutically used psychedelics and needed in some patients such as effects that are longer-lasting, different in quality and potentially more suitable than existing substances in some patients and complementing or substituting existing substances.

### SUMMARY OF THE INVENTION

The present invention provides for a method of inducing psychedelic states in an individual, by administering mescaline, a salt thereof, analogs thereof, or derivatives thereof to the individual, and inducing a psychedelic state in the individual.

The present invention provides for a method of therapy, by administering an intermediate “good effect dose” of mescaline, salt of mescaline, analogs thereof, or derivatives thereof to an individual, and inducing positive acute drug effects that are known to be associated with more positive long-term responses in psychiatric patients.

The present invention provides for a method of therapy, by administering an “ego-dissolution” dose of mescaline, a salt of mescaline, analogs thereof, or derivatives thereof to an individual, and providing the experience of ego-dissolution.

### DESCRIPTION OF THE DRAWINGS

Other advantages of the present invention are readily appreciated as the same becomes better understood by reference to the following detailed description when considered in connection with the accompanying drawings wherein:

FIG. 1 is a drawing of the chemical structure of mescaline and of possible derivatives (scalines, 3C scalines);

FIG. 2 is a table showing 5-HT receptor agonist activity of mescaline, psilocin (active metabolite of psilocybin), and LSD;

FIG. 3 is a table showing adrenergic and dopaminergic receptor binding affinity of mescaline, psilocin, and LSD;

FIGS. 4A-4H are graphs of the acute subjective effects of mescaline compared with psilocybin and LSD, showing any drug effect (4A), good drug effect (4B), bad drug effect (4C), stimulated (4D), anxiety (4E), nausea (4F), visual perception change (4G), and auditory perception change (4H);

FIGS. 5A-5H are graphs of additional acute subjective effects of mescaline compared with psilocybin and LSD, showing sounds influenced what I saw (5A), changed time perception (5B), ego dissolution (5C), gained insight (5D), talkative (5E), open (5F), trust (5G), and focus (5H);

FIGS. 6A-6B are graphs of the acute alterations of mind induced by mescaline and compared with psilocybin and LSD, with FIG. 6A showing six parameters, and FIG. 6B showing additional parameters; and

FIGS. 7A-7D are graphs of the acute cardiovascular effects of mescaline compared with psilocybin and LSD, showing systolic blood pressure (7A), diastolic blood pressure (7B), heart rate (7C), and body temperature (7D).

### DETAILED DESCRIPTION OF THE INVENTION

The present invention provides for a method of inducing psychedelic states by administering mescaline, a salt thereof, analogs thereof, or derivatives thereof in a controlled medical/psychological setting to an individual and inducing a psychedelic state for treating various medical conditions. Mind alterations can be induced with the compositions herein for treating medical disorders similar to other psychedelics but with fewer unwanted side effects as described below.

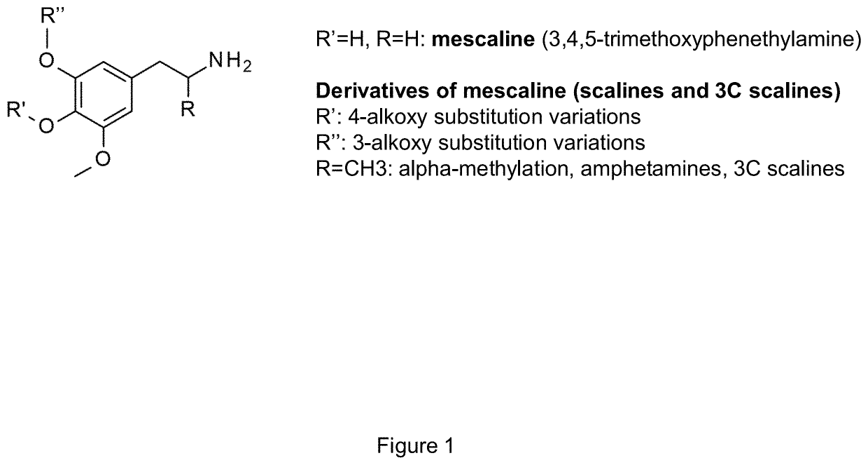

The structure of mescaline and possible sites of chemical modification leading to analogs or derivatives of mescaline are shown in FIG. 1. In the compound of FIG. 1, R is hydrogen, methyl, or ethyl, and

R′ is

C₁-C₅ branched or unbranched alkyl with the alkyl optionally substituted with F₁-F₅ fluorine substituents up to a fully fluorinated alkyl,

C₃-C₆ cycloalkyl optionally and independently substituted with one or more substituents such as F₁—F₅ fluorine and/or C₁-C₂ alkyl,

(C₃-C₆ cycloalkyl)-C₁-C₂ branched or unbranched alkyl optionally substituted with one or more substituents such as F₁-F₅ fluorine and/or C₁-C₂ alkyl, or

C₂-C₅ branched or unbranched alkenyl with E or Z vinylic, cis or trans allylic, E or Z allylic or other double bond position in relation to the attached ether function, where any of the carbons of the branched or unbranched alkenyl substituent is optionally substituted independently with one or more C₁-C₂ alkyl, with F₁-F₅ fluorine or with D₁-D₅ deuteron substituents.

The mescaline is synthetic or a comparable plant-derived purified extract. The mescaline can be mescaline hydrochloride as shown in the EXAMPLES, or any other salt thereof.

Mescaline can be used in doses from 1-800 mg. Specific doses can be used to provide different effects, which are further described in EXAMPLE 2. For example, a micro dose of mescaline can be 1-100 mg, a dose of 200 mg can be a low dose, a dose of 300-400 mg can be a moderate to medium-high dose, a dose of 500 can be a medium to high dose, and a dose of 800 mg can be a high to very-high dose. More specifically, and as further described below, a micro dose (1-100 mg) can induce no to minimal subjective effects and is equivalent to <20 μg of LSD base, a low dose (100-200 mg) can induce mild psychedelic effects and is equivalent to 20-40 μg of LSD, a moderate to medium dose (300-400 mg) can induce a moderate to medium strong psychedelic experience with mainly positive drug effects and is equivalent to 60-80 μg of LSD, a medium to high dose (500 mg) is equivalent to 100 μg of LSD base and induces a full “good effect” psychedelic response with mainly positive drug effects and moderate ego-dissolution and a moderate risk of producing anxiety, and a high dose (800 mg) is equivalent to 150-200 μg of LSD base and induces a full and very strong psychedelic response including marked “ego-dissolution” and has a high risk of producing anxiety.

Therefore, the present invention provides for a method of dosing and treating patients with mescaline, by administering mescaline, an analog thereof, or a derivative thereof at a specific dose defined as a micro dose, low dose, moderate dose, medium-high dose, high dose, or very high dose and producing positive subjective acute effects that are known to be associated with more positive long-term outcomes and minimizing negative acute effects.

The psychoactive properties of the serotonergic hallucinogen mescaline have likely been familiar to indigenous American tribes for more than 5000 years (51, 64, 65). Mescaline is a widespread cactus alkaloid found in particularly high concentrations in the buttons of the peyote cactus (Lophophora wiffiamsii) and the San Pedro cactus (Trichocereus pachanoi). Its psychoactive effects were scientifically discovered over more than a century ago by Arthur Heffter (66).

The use of mescaline within the present invention is distinct from the traditional religious/ritual use (Navajo, Native American Church of North America or shamanic uses in Latin America) of plants (peyote, San Pedro cactus, or related plants (67)) containing mescaline and with the purpose of mescaline to act as “entheogen” or “pant teacher” or “access to the divine” or “realms of Spirit” or as spiritual or cognitive tool to enhance spiritual experiences and embedded in a religious context (68, 69). In such a church/religious context, peyote has been or may still be effectively used in peyotists with drinking problems and has been considered to be safe (48, 50, 70). Other reported applications by Native Americans included rather poorly defined uses as a healing tool for snakebites, burns, wounds, fever, or “strength in walking” and others (48). Although, potentially prior therapeutic use, this use of plant mescaline is certainly not a medical treatment as the specified use of pharmaceutical mescaline in psychotherapy defined in the present invention.

Early reports of mescaline use did not represent clinical experimental studies and did not use valid control conditions, blinding, randomization, exact dosing, or outcome assessments using valid methods in contrast to the studies reported as part of the present invention (EXAMPLES 1 and 2).

The present invention includes the description of a clinical study comparing the acute effects of a defined oral dose of mescaline hydrochloride with those of psilocybin, LSD, and placebo as well as a dose-response study of EXAMPLE 1. The present invention newly documents an overall similar acute positive subjective drug effect profile for mescaline compared with psilocybin and LSD. The psychedelic effect profile induced by mescaline in the present study and overall similar to that induced by LSD or psilocybin is known to be predictive of therapeutic beneficial long-term effects not only in patients but also in healthy persons (7, 15, 56, 58, 71, 72). Thus, the present invention also describes a method of using mescaline in healthy subjects to derive positive acute effect profiles as shown in healthy subjects in the present study and known to be correlated with beneficial long-term effects and thus resulting in therapeutic effects in patients. Some of these effects including enhanced feelings of openness, trust, and gained insight were documented for mescaline in the present invention.

The present invention also provides for a method of inducing a psychedelic state in an individual that is longer-lasting than that induced by psilocybin and exhibiting partly different adverse effects and is therefore different in quality and potentially more suitable than existing substances in some patients and complementing or substituting existing substances. As EXAMPLE 1 shows, effects of mescaline were similar to those of psilocybin and LSD but typically longer lasting and with a lower and attenuated peak response at the doses used. Higher doses of mescaline can be used to reach similar peak effect as with psilocybin and LSD with a longer duration of action.

Psychedelics can be used to assist psychotherapy typically at acutely psychoactive doses and doses, for many indications including anxiety, depression, addiction, personality disorder, and others and can also be used to treat other disorders such as cluster headache, migraine, and others (1, 2, 4, 9, 10, 13, 14, 17, 18, 73, 74). Psychedelic-assisted psychotherapy includes a defined process distinct from using psychedelics as recreational substances or within a religious context. Patients meet with therapists for several preparatory sessions, then the psychedelic is administered once or twice, typically a few weeks apart, and integration sessions are conducted after sessions (63). Thus, mescaline within the present invention is to be used similarly to be effective.

Similar to the use of other psychedelics to assist psychotherapy, relatively high doses of mescaline that are expected to induce a psychedelic response are used within the present invention and the studies testing it in human subjects in EXAMPLES 1 and 2.

The induction of an overall positive acute response to the psychedelic is critical because several studies showed that a more positive experience is predictive of a greater therapeutic long-term effect of the psychedelic (9, 10, 15). Even in healthy subjects, positive acute responses to psychedelics including LSD have been shown to be linked to more positive long-term effects on well-being (75, 76).

The present invention tests one moderate dose of mescaline of 300 mg in human subjects in EXAMPLE 1 and in comparison, with psilocybin and LSD with the aim of inducing a positively experienced psychedelic state.

A higher dose of 500 mg can be used in an extension of EXAMPLE 1 and also in comparison with psilocybin and LSD using the same design as study EXAMPLE 1.

Second, lower and higher doses of mescaline are tested to further characterize the ideal dose of mescaline aiming for a maximum of positive over negative acute effects and an optimized psychedelic response (in study 2 in EXAMPLE 2).

Third, the use of mescaline compared to other substances within the present invention aims at having an additional substance at hand in patients who did not respond adequately to other substances which could include a response that was too low or too high or adverse and necessitating a change in substance to be used. This is a common approach in medicine where often a medication within a class of medications that resulted in negative effects or an insufficient response is replaced by another medication from the same class. Similarly, the present invention replaces psilocybin or LSD with mescaline in psychedelic-assisted therapy if needed.

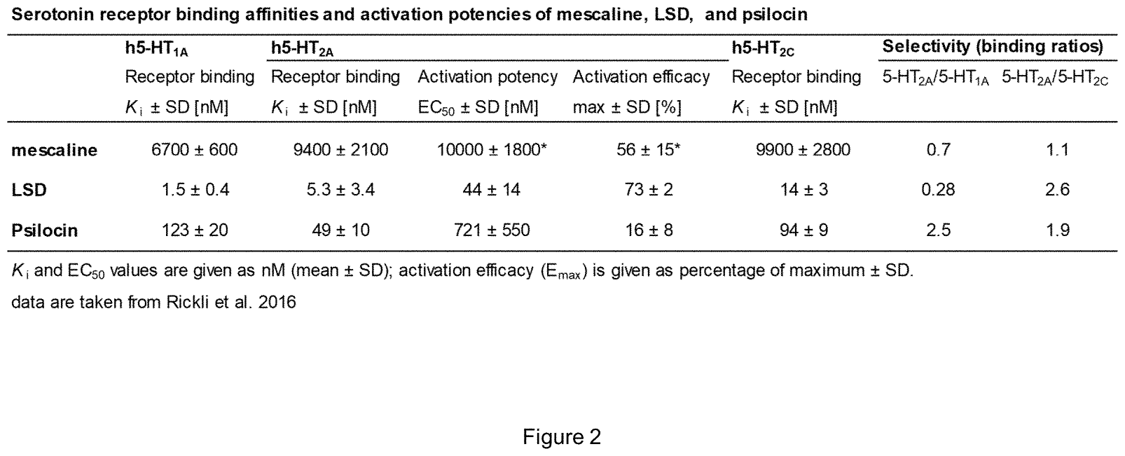

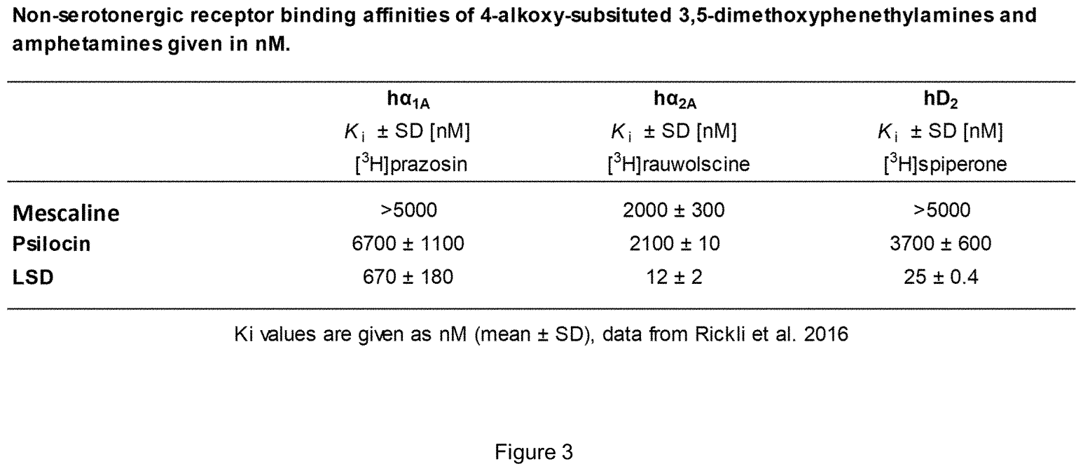

Mescaline is a classic serotonergic psychedelic. However, chemically, mescaline is a phenethylamine unlike LSD and psilocybin. Pharmacologically, LSD, psilocybin and mescaline are all thought to induce their subjective psychedelic effects primarily via their common stimulation of the 5-HT_{2A} receptor. However, there are differences in the receptor activation profiles between the substances that may induce different subjective effects. LSD potently stimulates the 5-HT_{2A} receptor but also 5-HT_{2B/C}, 5-HT₁ and D₁₋₃ receptors (FIG. 2). Psilocin (the active metabolite present in the human body derived from the prodrug psilocybin) also stimulates the 5-HT_{2A} receptor but additionally inhibits the 5-HT transporter (SERT). Mescaline binds in a similar rather low concentration range to 5-HT_{2A}, 5-HT_{1A} and α_{2A} receptors (FIG. 2-3). In contrast to LSD, psilocybin and mescaline show no affinity for D₂ receptors (FIG. 3). While mescaline does not directly interact with dopamine receptors (62) there is conflicting older data on potential dopaminergic effects. Effects in cats could be antagonized with the dopamine antagonist haloperidol (77). In contrast and as expected, mescaline generalized to LSD and psilocybin and other serotonergic hallucinogens in rat discrimination studies and this effect could be antagonized with 5-HT_{2A} receptor blockers but not dopamine receptor blockers including haloperidol (78). Enhancement of acoustic startle by mescaline in rats was blocked by serotonin 5-HT₂ receptor antagonists but not by a 5-HT₁ antagonist (79). Mescaline blocked the action of catecholamines potentially in line with its interaction with the adrenergic alpha₂ receptor (80). However, the adrenergic properties and effects in comparison with other psychedelics are not clear. Antipsychotics with antagonistic effects on serotonin 5-HT_{2A} and dopamine receptors blocked acute responses to mescaline in psychiatric patients (81). However, the study is methodologically invalid to derive conclusions.

Taken together, LSD can have greater dopaminergic activity than psilocybin and mescaline, psilocybin may have additional action at the SERT. Mescaline and derivatives do not interact with the SERT.

Furthermore, there can also be differences in the activation of the intracellular second-messenger pathways produced by different 5-HT_{2A} receptor agonists which could also result in differential effects of different psychedelics. Thus, mescaline can also have different effects compared with LSD or psilocybin base on such yet now well-defined differences down-stream activation patterns (54, 82). Finally, there can also be differences in brain wide circuit activations and neuronal activation patterns as to be further defined in future optogenetics and/or brain imaging studies that can show distinct properties of different serotonergic compounds including mescaline compared with LSD and psilocybin (83).

Within the present invention, the clinical study (EXAMPLE 1) tested whether similarities and differences in the pharmacological profiles of mescaline, psilocybin and LSD in vitro translate into similar and/or different subjective effects in humans. Because the primary action of all these hallucinogens is the activation of the 5-HT_{2A} receptor and based on preliminary data (36), there are likely not marked differences in the subjective alterations of consciousness acutely induced by these substances making them all suitable as psychedelic therapeutics. Nevertheless, there are differences in the binding potencies of the three substances at their primary target. Therefore, it was expected to document small differences that would translate into advantages of one substance over the other in selected clinical situations. For example, psilocybin interacts with the SERT, while mescaline does not (62). Because serotonin is involved with hyperthermia and MDMA, which also interacts with the SERT (84) can induce fatal hyperpyrexia (85), psilocybin can also lead to greater thermogenic responses compared with mescaline in humans. Indeed, the present invention (Example 1) showed reduced thermogenic effects of mescaline compared to psilocybin.

LSD binds most potently to the 5-HT_{2A} receptor followed by psilocybin and mescaline (62) (FIG. 2). Mescaline is the least potent of all classic hallucinogens. It is about 1000-3000 times less potent than LSD and around 30 times less potent than psilocybin (86) consistent with in vitro data (62) (FIG. 2). The binding potency of hallucinogens in vitro correlates with their potency to acutely induce alterations of subjective effects in humans (87). Importantly, mescaline has very strong hallucinogenic properties in humans despite its low potency at the 5-HT_{2A} receptor (51) but relatively high doses are needed to produce subjective responses. Additionally, there are differences in the duration of the effects among these psychedelics. Mescaline can have a delayed onset of action possibly due to a slow brain penetration (88). The subjective effect duration of a moderate mescaline dose is 10-12 hours, therefore similar to that of a moderate dose (0.1 mg) of LSD and exceeding the duration of acute psilocybin effects (4-6 hours) (86). These previously documented preliminary effect duration times for the individual substances were tested within the present invention using a valid modern clinical study directly comparing the effects of the three substances in the same subjects and using sensitive measures.

Taken together the pharmacological profiles of LSD, psilocybin and mescaline show some differences but it is not clear whether these are reflected by differences in their psychoactive profiles in humans. Furthermore, mescaline has an old tradition of use but has not been compared with the more recently investigated psychedelics LSD and psilocybin and its therapeutic use potential has not been defined (51).

The compounds of the present invention can be used in assisting psychotherapy or treatment for many different indications, including anxiety disorder, anxiety associated with life-threatening illness, depression, addiction including substance use disorder and impulse control disorder (behavioral addiction), personality disorder, compulsive-obsessive disorder, post-traumatic stress disorder, eating disorder, cluster headache, migraine, and any other disorder where psychedelic psychotherapy or therapy can be useful.

The compounds of the present invention can be used when the individual has an insufficient therapeutic response or adverse effects after the use of other psychedelics substances and the methods herein can be used as a second-line treatment. The compounds of the present invention can be used when the individual has a need for a qualitatively different psychedelic response after the use of other psychedelics substances and the method can be used as an alternative treatment option. The individual can have a need for a more attenuated response, with a slower onset of the psychological, or physiological response of the psychedelic (attenuated and prolonged response) compared with other psychedelics such as psilocybin or LSD and the inducing step provides an effect of less nausea and vomiting than psilocybin, less cardiovascular stimulation than psilocybin, reduced thermogenic acute effects compared with psilocybin, less bad drug effects including anxiety, fewer or less intensive headaches than psilocybin, an overall slow and attenuated effect onset compared with psilocybin, reduced peak response at longer effect duration and overall effect than comparable treatment options such as psilocybin, an overall intensive subjective experience while exhibiting a favorable acute adverse effects profile, and/or combinations thereof.

Inducing a psychedelic state with the compounds of the present invention can reduce the risk of nausea or vomiting within a psychedelic treatment session, reduce the risk of cardiovascular stimulation within a psychedelics treatment session, or increase feelings of trust and openness beneficial in enhancing the therapeutic alliance and catalyze the effects of psychotherapy for any indication. Inducing a psychedelic state can also produce an inward oriented focus of attention and subjective insight to enhance psychotherapy for any indication, or induce neuroregenerative processes beneficial in medical conditions such as, but not limited to, Alzheimer's disease, dementia, predementia, or Parkinson's disease.

The present invention provides for a method of therapy, by administering an intermediate “good effect dose” of mescaline, salt of mescaline, analogs thereof, or derivatives thereof to an individual, and inducing positive acute drug effects that are known to be associated with more positive long-term responses in psychiatric patients. This is further described in EXAMPLE 2.

The present invention provides for a method of therapy, by administering an “ego-dissolution” dose of mescaline, a salt of mescaline, analogs thereof, or derivatives thereof to an individual, and providing the experience of ego-dissolution. This is further described in EXAMPLE 2.

### Example 1: Clinical Study I: Direct within-Subject Comparison of Acute Effects of Single Oral Doses of Mescaline (300 mg), Psilocybin (20 mg), and LSD (100 μg) in Healthy Subjects

The study directly compared the acute effects of single doses of three different classic hallucinogens using a cross-over design. The primary objective of this study was the comparison of the quality of subjectively altered states of consciousness induced by mescaline, psilocybin, and LSD. It was hypothesized that all three substances would induce similar psychedelic states measured using visual analog scales and the 5 dimensions of altered states questionnaire (5D-ASC). It was expected that mescaline would produce a state of altered consciousness lasting longer than after the administration of psilocybin. Some differential effects were expected regarding subjective effect qualities, autonomic responses, and adverse effects.

Study design: The study used a randomized, double-blind, double-dummy, cross-over design with four conditions.

Study intervention: Each subject participated in 4×25-hour study sessions. Conditions were 1) 100 μg LSD, 2) 20 mg psilocybin, 3) 300 mg mescaline and 4) placebo. Order was randomized and balanced with washout periods of at least 10 days between study days.

LSD: LSD is a very potent partial 5-HT_{2A} receptor agonist (62, 74). LSD also stimulates 5-HT₁ receptors, adrenergic α₁ receptors and dopaminergic D₁₋₃ receptors (62, 89). An intermediate dose of 100 μg was be used. A 100 μg dose induces intermediately strong and typical LSD effects in healthy subjects with peak responses at 3 hours and lasting for 8 hours (3, 45, 55, 90, 91).

Psilocybin: Psilocybin is a 5-HT_{2A} receptor agonist and 5-HT transporter inhibitor. Most of the psilocybin effects are mediated by the 5-HT_{2A} receptor (92) but psilocybin also activates the 5-HT system via 5-HT transporter inhibition (62) which can produce additional MDMA-like empathogenic effects. The present study used 20 mg of psilocybin, a dose that was previously used in healthy subjects with good tolerability (59, 93, 94). A similar dose has also been used in patients (8, 9, 74). Psilocybin has been used in similar experimental studies by several groups in human subjects (16, 17, 59, 94-96). It was expected that the 20 mg psilocybin dose would produce a similarly strong effects as the 100 μg dose of LSD.

Mescaline: Mescaline is a nonselective serotonin receptor agonist and binds to the 5-HT_{2A} receptor which has ensured its categorization as a classic hallucinogen, although it does so with lower potency and higher activity compared to LSD (54, 62, 78). Unlike LSD and psilocybin, mescaline shows equally high affinity for 5-HT_{1A} and adrenergic α_{2A} receptors (51, 97) and is not classified as an indolalkaloid, but as a phenethylamine, structurally resembling stimulants like amphetamine and catecholamines like norepinephrine and dopamine, which are involved in processes of both neurotransmission and neurotoxicity (98). Mescaline is 1000-3000 times less potent than LSD and around 30 times less potent than psilocybin, requiring a relatively high dose of about 300 mg to induce a full psychedelic experience (51, 86). The psycho-pharmacological effects of mescaline are considered prototypical despite its stimulant-like chemical structure (86). Indigenous tribes across the northern and southern parts of America have used mescaline for centuries for ethnomedical purposes (50, 69, 70). Regular mescaline consumption due to these ceremonies has not been associated with significant harm, but some adverse effects like psychotic episodes and transient anxiety have been reported [Halpern, 2005 #5872).

Clinical studies on the effects of mescaline in man are scarce. There is no modern data. Physiological and psychological effects were reported to set in approximately 30 minutes after oral administration of 500 mg mescaline hydrochloride, to peak at 4 hours and to last 12 to 14 hours (46). The average half-life of mescaline is approximately six hours (46). Mescaline shows cross-tolerance with LSD in human studies (99, 100). Termed “psychotomimetic” in previous decades due to its similarity with acute psychotic states, mescaline provokes hallucinative, stuporous or paranoid appearances (39, 40, 101). It has been reported that LSD and mescaline tend to induce a wider synesthetic spectrum compared to psilocybin (102). Early studies indicate that LSD can elicit auditory-visual, music-visual, color-gustatory, color-auditory, and music-olfactory synesthesia (36, 101, 103, 104), whereas mescaline is reportedly more likely to induce haptic-visual, auditory-visual, kinesthetic-visual, and algesic-color synesthesia (103, 105). Psilocybin on the other hand has only been shown to induce auditory-visual synesthesia (36, 106). Doses of 200-400 mg mescaline sulfate have been reported to induce hallucinations lasting for about 10-12 h (107). The oral dose used in this study (300 mg) has been described as a moderate dosage generating a full hallucinogenic experience (51, 101) and is expected to induce acute subjective effects lasting up to 12 hours. It is assumed that the intensity of subjective effects caused by 300 mg of mescaline will correspond to the intensity of the moderate LSD and psilocybin doses used in this study. Adverse effects like psychotic episodes and transient anxiety due to depersonalization and derealization have been reported (50, 70). Adverse psychological effects following hallucinogen intoxication can be alleviated with talk-down strategies or, if necessary, benzodiazepines (108, 109). Mescaline provokes physiological effects similar to epinephrine and norepinephrine: Tachycardia, hypertension, increase in temperature, perspiration, nausea, dizziness, pupil dilation, tremor, restlessness, and dry mouth (39, 110). An analysis of 31 cases of mescaline consumers that were registered between 1997 and 2008 at the California Poison Control System database revealed that the most frequently reported effect was hallucinations, followed by tachycardia, agitation, and mydriasis (48). Interestingly, the frequently reported adverse effect of vomiting could not be confirmed in this study. The authors assumed that vomiting is more likely due to the bitter taste of the plant rather than actual gastric effects of mescaline (48). This hypothesis contradicts early investigations done in the 1930s, which reported the common occurrence of initial nausea after subcutaneous injection of 300 mg (101). Mescaline, just like all serotonergic hallucinogens, has been continuously reported neither to cause any physiological harm nor to trigger addictive behavior (59, 111). Furthermore, lifetime use of hallucinogens is not associated with increased mental health issues (112). It has been assumed that the abuse potential of the substance is very low due to bitter taste, nausea and low potency, resulting in a slow onset of subjective effects (86). On the contrary, it has been suggested that hallucinogens like mescaline can hold anti-addictive properties and that these substances might be safe and effective tools to support drug dependence recovery (113). The development of slow tolerance to repeated administration has been reported in animals (114).

Participants: The preliminary study sample shown and used herein in EXAMPLE 1 to illustrate and put into practice the present invention included healthy subjects (male and females). Inclusion criteria were: Age between 25 and 65 years old; Sufficient understanding of the German language; Understanding of procedures and risks associated with the study; Willing to adhere to the protocol and signing of the consent form; Willing to refrain from the consumption of illicit psychoactive substances during the study; Abstaining from xanthine-based liquids from the evenings prior to the study sessions to the end of the study days; Willing not to operate heavy machinery within 48 hours after substance administration; Willing to use double-barrier birth control throughout study participation; Body mass index between 18-29 kg/m2. Exclusion criteria were: Chronic or acute medical condition; Current or previous major psychiatric disorder; Psychotic disorder or bipolar disorder in first-degree relatives; Hypertension (>140/90 mmHg) or hypotension (SBP<85 mmHg); Hallucinogenic substance use (not including cannabis) more than 20 times or any time within the previous two months; Pregnancy or current breastfeeding; Participation in another clinical trial (currently or within the last 30 days); Use of medication that may interfere with the effects of the study medication; Tobacco smoking (>10 cigarettes/day); Consumption of alcoholic beverages (>20 drinks/week). Subjects were recruited via advertisement displayed on the website of the University of Basel. Mainly university students were included. Screening visits and sessions were performed in the Ambulatory Study Center, located in the Department of Clinical Research at the University Hospital of Basel. Screening procedure: Subjects were examined by a study physician. Basic health was ensured by general medical examination including medical history, physical examination, electrocardiogram, determination of body weight and blood chemistry and hematology analysis. Additionally, subjects were screened using a semi-structured clinical interview for DSM-V (115) to exclude those with a personal or first-degree relative axis I major psychiatric disorder (acute or past) or a history of drug dependence. Additionally, the ‘Self-screening Prodrome’ (116) was used to ensure early detection of psychotic tendencies. Axis I major psychiatric disorders also include addiction disorders. Informed consent: Subjects were informed about the study procedures and associated risks in advance through the written participant information.

Study Procedures

Psychometric Assessment

Subjective Effects Questionnaire (Visual Analog Scales, VAS): VAS was repeatedly used to assess subjective alterations in consciousness over time. Single scales were presented as 100 mm horizontal lines marked with “not at all” on the left and “extremely” on the right. The following VAS items were used: “any drug effect”, “good drug effect”, “bad drug effect”, “stimulated”, “anxiety”, “nausea”, “alteration of vision”, “alteration of hearing”, “sounds seem to influence what I see”, “alteration of sense of time”, “the boundaries between myself and my surroundings seem to blur (ego dissolution)”, “I am having insights into connections that previously puzzled me”, “talkative”, “open”, “trust”, and “insight”. Scales were administered before and repeatedly after substance administration.

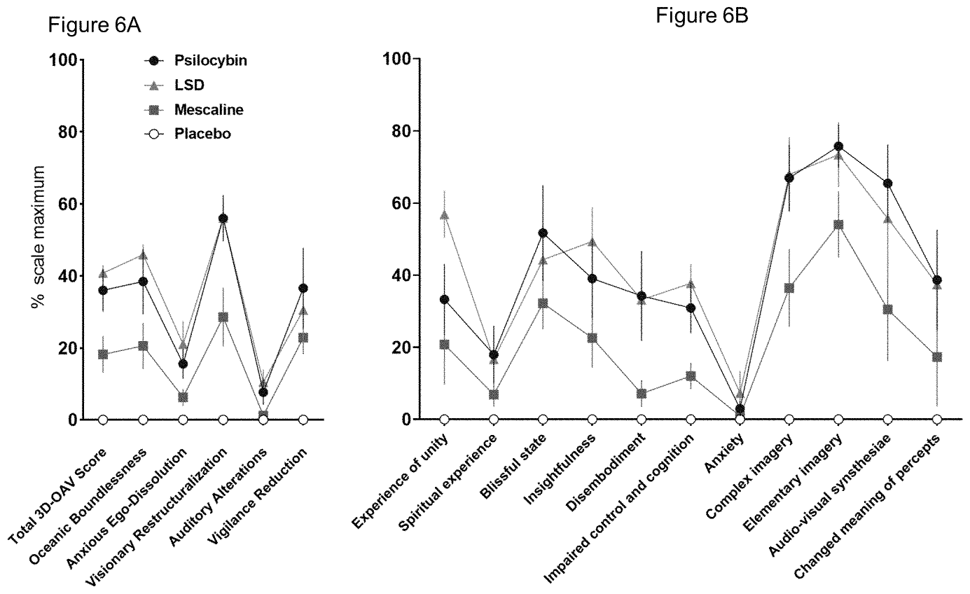

5-Dimensional Altered States of Consciousness (5D-ASC): The 5-dimensional Altered States of Consciousness (5D-ASC) Scale is a visual analog scale consisting of 94 items (117, 118). The instrument contains five main scales (FIG. 6A) and 11 newer subscales (FIG. 6B) assessing mood, anxiety, derealization, depersonalization, changes in perception, auditory alterations, and reduced vigilance. The scale is well-validated (118). The 5D-ASC scale was administered once at the end of the session and subjects were instructed to retrospectively rate peak alterations that have been experienced during the study session. Each item of the scale is scored on a 0-100 mm VAS. The attribution of the individual items to the subscales of the 5D-ASC was analyzed according to (117, 118) and as shown in FIGS. 6A-6B. The scale was be administered once at the end of each test session.

Autonomic measures: Blood pressure, heart rate, and body temperature were recorded at baseline and repeatedly throughout the session. Blood pressure (systolic and diastolic) and heart rate were measured with an automatic oscillometric device. Body temperature was measured with an ear thermometer.

Adverse effects (list of complaints): The list of complaints (LC) consists of 66 items offering a global score measuring physical and general discomfort (119). The LC list was administered 12 hours after administration of the drug with reference to complaints throughout the entire session.

The study included additional outcomes not discussed here.

Substance preparation and quality control: Mescaline was prepared as capsules containing 100 mg of analytically pure mescaline (ReseaChem GmbH, Burgdorf, Switzerland) and mannitol filler. Psilocybin was prepared as capsules containing 5 mg of analytically pure psilocybin (ReseaChem GmbH, Burgdorf, Switzerland) and mannitol filler. LSD was prepared as an oral solution containing 100 μg of analytically pure LSD (Lipomed AG, Arlesheim, Switzerland) in 1 ml of ethanol. All three substance formulations plus matching placebos were prepared by a GMP facility (Apotheke Dr. Hysek, Biel, Switzerland) according to GMP guidelines. LSD-placebo solutions consisted of only ethanol, psilocybin and mescaline-placebo capsules consisted of only mannitol. All placebos were prepared by the same GMP facility and looked identical to the verum preparations to ensure proper blinding. The study used a double-dummy design which means that every patient received a psilocybin/mescaline placebo with the LSD verum, and an LSD placebo with the mescaline or psilocybin verum. Randomization, packaging, labelling, and quality control (QC) including stability tests were handled by the GMP facility. Subjects and study personnel involved in supervising the session were blinded to treatment order that was balanced.

Results of the Clinical Study I (Example 1)

A key goal of the present study as part of the present invention was to measure in humans the acute effects of mescaline that are considered to be predictive of therapeutic potential in patients.

Positive acute effects of a psychedelic on the 5D-ASC scale and other scales have previously been documented to correlate with beneficial therapeutic outcomes in patients. Specifically, psilocybin reduced alcohol or nicotine use or in dependent patients over months and positive outcomes were correlated with the intensity of positive acute mystical-type experiences that the subject reported (13, 15, 17). Improvements five weeks after psilocybin treatment of patients with treatment-resistant depression were predicted high ratings of acute effects of pleasurable ego-dissolution (OB) including feelings of bliss and unity and low scores of anxious ego-dissolution (AED) in the 5D-ASC questionnaire (7). Similarly, long-term symptom improvement in patients with anxiety and depression correlated with greater scores of acute mystical-type experiences (9, 10).

Taken together, acute alterations of self-processing, positively experienced self-dissolution with feelings of connectedness or oneness with the world (similar to mystical-type experiences) have generally been associated with positive long-term treatment outcomes with the use of psychedelics in controlled settings. Positive long-term effects were even noted in healthy subjects after LSD or psilocybin use in a safe setting (75, 76).

The present invention, for the first time, demonstrated positive acute effects for mescaline that are very similar to those linked to positive long-term outcomes after LSD or psilocybin treatment in patients (63).

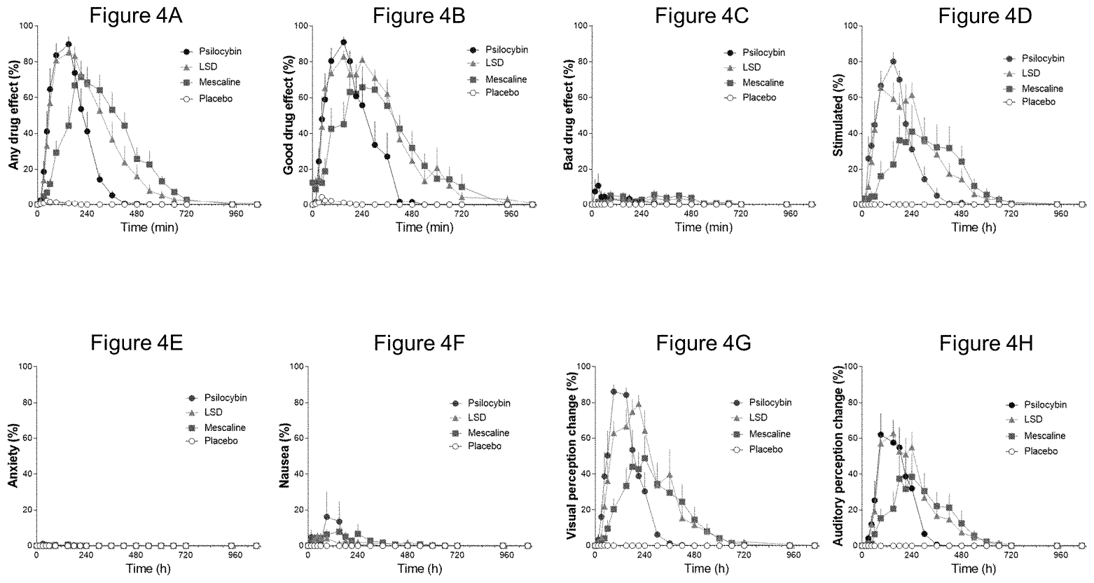

FIGS. 4A-4H show the acute subjective effects of psilocybin, LSD, mescaline, and placebo on the VAS (any drug effect, good drug effect, bad drug effect, stimulated) over the time of one session in six healthy volunteers. Effects of psilocybin lasted less long compared with LSD and mescaline (FIG. 4A). Effects of LSD and mescaline lasted similarly long at the doses used (FIG. 4A). Effects of mescaline took longer to peak compared to those of psilocybin and LSD and were lower than those of psilocybin and LSD at the doses used (FIGS. 4A and 4B). While the peak effect was greater after psilocybin and LSD compared with mescaline, the area under the effect over time curve was similar after psilocybin (higher Eₘₐₓ but shorter duration) and mescaline (lower Eₘₐₓ but longer duration) and greater after LSD (both high Eₘₐₓ and long time-of-action) at the doses used (FIGS. 4A and 4B).

Qualitative effect profiles were similar with all active substances having mostly positive (FIG. 4B) over negative effects (FIGS. 4C and 4E). Bad drug effects were low, and peaks lower for LSD and mescaline compared with psilocybin (FIG. 4C).

Both psilocybin and LSD produced greater stimulation than mescaline at the doses used (FIG. 4D).

None of the substances produced relevant anxiety (FIG. 4E).

Nausea was present with all substances in a few subjects and highest with psilocybin, followed by LSD and mescaline, which produced the lowest nausea ratings (FIG. 4F).

Both visual (FIG. 4G) and auditory (FIG. 4H) perception were altered markedly and with highest ratings for psilocybin and LSD. Mescaline produced lower peak effects than both psilocybin and LSD (FIGS. 4G and 4H). Area under the effect-time curve values were similar for psilocybin and mescaline and greater for LSD. Thus, mescaline produced lower perceptual alterations but lasting longer compared with psilocybin (FIGS. 4G and 4H).

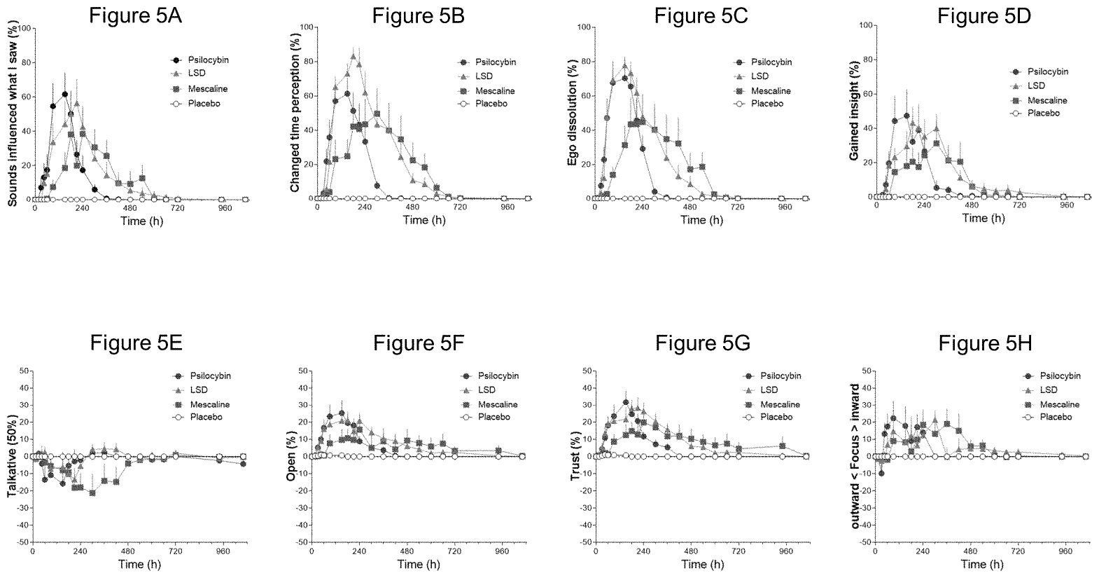

All substances induced synesthesia as indicated by high ratings of “sounds influenced what I saw” with highest ratings after psilocybin and LSD and lower ratings after mescaline (FIG. 5A).

Time perception was acutely altered by all substances with trends toward lower changes induced mescaline compared with LSD and indicating more attenuated changes and a greater presence in the “here and now” with mescaline) (FIG. 5B).

Ego-dissolution was mostly increased by LSD and psilocybin with again lower ratings after administration of mescaline and rated on the VAS labeled “the border between me and my surroundings seemed to blur” (FIG. 5C). Ego-dissolution is a typical phenomenon induced by full doses of psychedelic substances and indicating a full psychedelic experience. The ratings indicate that higher doses of mescaline than the one used (300 mg) can be used to induce a full peak psychedelic response (FIG. 5C).

Gains in insight ratings were relatively similar across substances (FIG. 5D).

All substances also similarly tended to reduce talking during the first hours with mescaline producing the longest effect (FIG. 5E).

Openness was increased with all substances with mescaline having the smallest peak effect but a longer lasting effect compared with psilocybin (FIG. 5F).

Trust was similarly increased by all substances (FIG. 5G).

Attention was oriented more inward during the session after administration of all substances (FIG. 5H).

Taken together, effects of mescaline were similar to those of psilocybin and LSD but typically longer lasting and with a lower and attenuated peak response at the doses used. Higher doses of mescaline can be used to reach similar peak effect as with psilocybin and LSD with a longer duration of action. This is tested within the present invention by increasing the dose of mescaline from 300 mg to 500 mg in an additional cohort of healthy subjects while keeping the doses of psilocybin and LSD at the level used.

FIGS. 6A-6B show effects of psilocybin, LSD, mescaline, and placebo on the 5D-ASC scale. Data are mean±SEM values from 6 subjects. Effects are peak responses of the substances retrospectively rated 12 hours after drug administration. At the doses used, mescaline (300 mg) produced approximately 50% of the total alterations of consciousness (total 3D-OAV score, FIG. 6A) ratings observed with LSD (100 μg) or psilocybin (20 mg). LSD and psilocybin had equally strong overall peak effects (total 3D-OAV scores) at the doses used and produced overall similar score ratings on the different ASC sub-scores (FIGS. 6A and 6B). The overall relative effects of mescaline on the scale were similar but lower to those of LSD or psilocybin on the different dimensions of the scale and subscales. While not tested in the present study so far, a dose of 500-600 mg of mescaline, which is 1.67- to 2-fold as high as the dose used, is be expected to produce overall similar effects to LSD or psilocybin base on the present data. However, the 5D-ASC compared peak responses and the duration of the experience is longer with mescaline than with LSD or psilocybin.

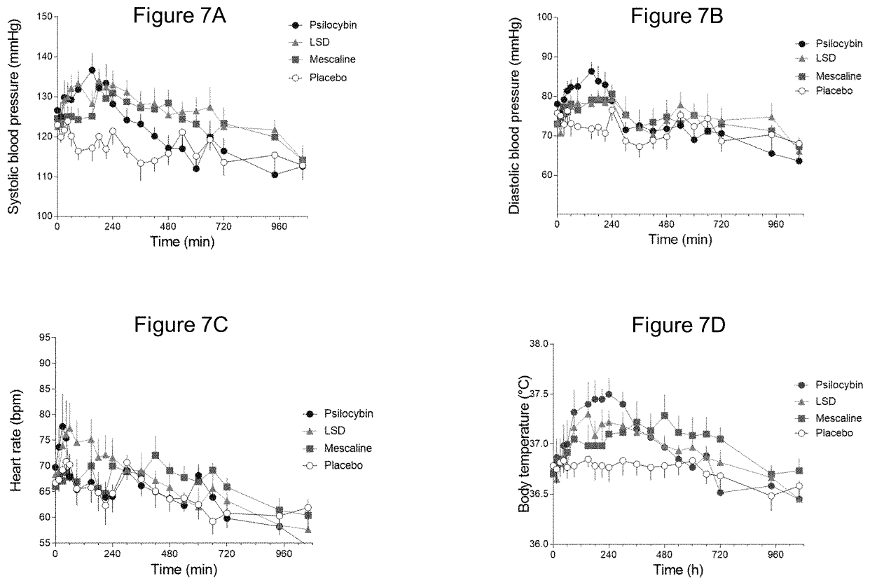

FIGS. 7A-7D show the vital sign changes after administration of psilocybin, LSD, mescaline, and placebo. Data are “mean±SEM” values from 6 subjects. All active substances produced only relatively moderate increases in blood pressure (FIGS. 7A and 7B) and heart rate (FIG. 7C) compared with placebo. There were only minimal differences in the autonomic effects between active compounds. Psilocybin produces a more pronounced and shorter-lasting increase in blood pressure (FIGS. 7A and 7B) and body temperature (FIG. 7D) than LSD or mescaline. Increases in blood pressure (FIGS. 7A and 7B), heart rate (FIG. 7C), and body temperature (FIG. 7D) after mescaline tended to be attenuated compared with both psilocybin and LSD and tended to last longer than those of psilocybin and similar to LSD. Overall, effects of mescaline on heart rate tended to be lower than those of psilocybin and potentially also those of LSD at the doses used. However, more data is needed to confirm this finding with statistical tests.

Mescaline produced similar adverse effects and similar total LC scores to LSD and psilocybin on the LC and at the doses used. The mean total LC scores were 6.8, 5.4, 8.8, and 0.8 after psilocybin, LSD, mescaline, and placebo, respectively in six human volunteers. Thus, the overall tolerability of acute mescaline administration was overall similar to that of LSD or psilocybin.

### Example 2 (Study II): Clinical Dose Finding Study Using Different Single Oral Doses of Mescaline Hydrochloride in Healthy Subjects

The present invention also relates to the use of specific doses of mescaline to produce defined subjective drug effects in helping to treat medical conditions. Because no dose-response data was available on mescaline, the present invention also includes a dose-response study in healthy subjects to define acute effects of mescaline across different doses.

To define the doses of mescaline within the present invention a dose-finding or “dose response study” is being conducted in healthy human subjects. The study goal is to characterize the dose-response relationship in mescaline-induced altered states of consciousness. The study population consists of healthy subjects (male and female). The study design is double-blind, placebo-controlled, and cross-over. Mescaline is administered on the study days that are separated by at least 10 days in the following doses: 1) mescaline 100 mg, 2) mescaline 200 mg, 3) mescaline 400 mg, 4) mescaline 800 mg, and 5) placebo, in counterbalanced order. The primary endpoints are subjective effects (VAS, 5D-ASC) and tolerability (body temperature, blood pressure, heart rate, adverse effects). This study defines the doses of mescaline to induce alterations of consciousness and provides the amount of acute effect for each dose. This complements the study comparing mescaline at only one dose of 300 mg with LSD and psilocybin and provides a unique set of data to define the dose to be used in the present invention.

The dose-response study within the present invention provides for a method of dosing and treating patients with mescaline, by administering mescaline or an analog thereof at a specific dose defined below such as a micro dose, moderate dose, medium-high dose, high dose or very high dose and producing positive subjective acute effects that are known to be associated with more positive long-term outcomes and minimizing negative acute effects. Defined doses of the mescaline can be administered with specific acute effects defined for a dose and specific indications for defined doses of mescaline. The overall goal of the dose-response study within the present invention, using mescaline is to improve the “positive” over “negative acute subjective effect” response to this psychedelic. The method of mescaline dosing applies to indications where a positive experience after psychedelic use predicts the long-term effects such as in psychiatric disorders including (but not limited to) depression, anxiety, and addiction.

“Positive acute effects” as used herein refers primarily to an increase in subjective rating of “good drug effect” and may also include ratings of “drug liking”, “well-being”, “oceanic boundlessness”, “experience of unity”, “spiritual experience”, “blissful state”, “insightfulness”, any “mystical-type experience” and positively experienced “psychedelic effects”, and “aspects of ego-dissolution” if experienced without relevant anxiety.

“Negative acute effects” as used herein refers primarily to subjective ratings of “bad drug effect” and “anxiety” and “fear” and may additionally include increased ratings of “anxious ego-dissolution”, or descriptions of acute paranoia or states of panic an anxiety as observed by others.

The following dosing recommendations are defined within the present invention and will be further refined once more data becomes available.

A “micro dose” is a dose of a psychedelic not producing distinct acute subjective drug effects compared with placebo and in line with (120, 121). A micro dose of mescaline is 1-100 mg equivalent to 0.2-20 μg of LSD base. Such doses have no or minimal subjective acute effects but may have therapeutic effects in humans.

A dose of 200 mg of mescaline is a small dose that can be used as starting point in an individual with no experience or an expected high susceptibility or if a very small response is wanted in a patient. Such a small dose of 200 mg or even smaller doses (<200 mg) may also be useful when “micro dosing” is the goal and/or including repeated doses of mescaline that produce no or only minimal psychoactive effects. Such low doses may particularly be useful to treat disorders such as cluster headache or migraine with mescaline similar to the use of low doses of LSD in these disorders (122-127). A low or micro dose of mescaline is also useful when aiming to treat depression with a micro dose producing minimal acute effect but producing a therapeutic response similar as conceived for the use of low doses of other psychedelics in depression (128). A dose of mescaline hydrochloride of 200 mg is equivalent to 40 (25-50) μg of LSD.

A dose of mescaline of 300-400 mg is a moderate to medium-high dose useful in most cases as a starting dose or repeat dose in experienced persons and equivalent to doses of LSD of 60-80 (50-100) μg LSD base or 15-20 mg of psilocybin.

A dose of mescaline of 500 is a medium to high dose useful in patients with previous experience with a lower dose of mescaline or with experience with other psychedelics or in any patients where a very stronger effect is desired. This dose of 500 mg of mescaline would correspond to a dose of LSD of 100 μg of LSD base or 20 mg of psilocybin.

A dose of mescaline of 800 mg is a high to very-high dose useful in patients with previous experience with a lower dose of mescaline or with experience with other psychedelics or in any patients where a very strong effects are desired. This dose of 800 mg of mescaline would correspond to a dose of LSD of 150-200 μg of LSD base or 25-40 mg of psilocybin.

Mescaline can be used to assist psychotherapy, typically at acutely psychoactive doses, for many indications including anxiety, depression, addiction, personality disorder, and others and can also be used to treat other disorders such as cluster headache, migraine, and others similar to psilocybin or LSD.

The induction of an overall positive acute response to the psychedelic is critical because several studies showed that a more positive experience is predictive of a greater therapeutic long-term effect of the psychedelic (9, 10, 15). Even in healthy subjects, positive acute responses to psychedelics such as LSD or psilocybin has been shown to be linked to more positive long-term effects on well-being (75, 76). A positive overall response similar to representative and therapeutically used doses of LSD of 100 μg or psilocybin of 20 mg was documented for mescaline (300 mg) within the present invention.

Moderate to high doses of mescaline of 300-500 are useful to enhance psychotherapy for most indications including anxiety, depression, compulsive obsessive disorder, eating disorder, post-traumatic stress disorder, addiction (alcohol, nicotine, behavioral, cocaine, amphetamines), anxiety associated with life-threatening illness, adjustment disorder, cluster headache, and migraine.

A high to very high dose of mescaline of 800 mg is particularly useful in cases where a very strong effect is desired. This includes patients were a higher degree of “ego dissolution” is targeted such as patients with cancer, pain, addiction with high tolerance such as opioid dependence and any other disorders such as personality disorder that may need high doses and high ego dissolution effects at the expense of greater acute anxiety and potentially greater adverse effects. Thus, a method of dosing mescaline at high to very high doses is appropriate for individuals experienced with lower doses of mescaline or other psychedelics and aiming for a more intense and ego-dissolving experience but also ready to risk experiencing greater anxiety when dealing with this state. Ego-dissolution as experience may be therapeutic in some indications namely in individuals with severe pain disorders, with cancer and/or in palliative care with the goal of being free of pain or at least not realizing somatic pain and the presence of the body or feeling out of the body during this experience. Ego-dissolution can also be a therapeutic experience in other disorders including personality disorder (narcissistic personality disorder) or as needed by psychiatric indications.

The psychedelics used in the methods of the present invention can be, but are not limited to, mescaline or any derivatives, any analogs or derivatives (scalines, 2C- or 3C-substances, FIG. 1, or prodrugs of mescaline salts thereof, analogs thereof, or homologues thereof.

Mescaline or related compounds of the present invention are administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus further determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compounds of the present invention can be administered in various ways. It should be noted that they can be administered as the compound orally as done in the example study and can be administered alone or as an active ingredient in combination with pharmaceutically acceptable carriers, diluents, adjuvants, and vehicles. The compounds can be administered orally, subcutaneously or parenterally including intravenous, transcutaneous, intramuscular, and intranasal administration. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses or a continuous dose over a period of several hours.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

To summarize, the specific uses of mescaline and its analogs in the context of substance assisted psychotherapy in humans within the present invention are described as follows: Mescaline can be used to assist and enhance any type of psychotherapy. A mescaline-assisted session can be used after conducting psychotherapy sessions in a person without mescaline. A mescaline-assisted session can be integrated in non-substance assisted psychotherapy. Mescaline can also be used after other psychedelics such as psilocybin or LSD or the empathogen MDMA have been used in a patient and resulted in insufficient responses or adverse effects. Thus, mescaline expands the range of possible substances to be used to assist psychotherapy.

Mescaline can also be preferred in some patients with expected adverse effects to other substances. For example, it may not be desired to use MDMA in some patients with an increased risk for specific adverse effects such as in patients with cardiovascular disease of arterial hypertension or genetic disorders such as malignant hyperthermia. In such patients, mescaline can be used instead of another psychedelic or MDMA to reduce the risk of adverse effects to other substances.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible considering the above teachings. It is, therefore, to be understood that within the scope of the appended claims, the invention can be practiced otherwise than as specifically described.

What is claimed is:

1. A method of inducing a psychedelic state in an individual, including the steps of:
administering a composition chosen from the group consisting of mescaline, a salt thereof, analogs thereof, and derivatives thereof to an individual; and
inducing a psychedelic state in the individual while reducing the risk of nausea or vomiting within a psychedelic treatment session and reducing the risk of cardiovascular stimulation within a psychedelic treatment session.

2. The method of claim 1, further including the step of treating a medical condition chosen from the group consisting of anxiety disorder, anxiety associated with life-threatening illness, depression, addiction, personality disorder, compulsive-obsessive disorder, post-traumatic stress disorder, eating disorder, cluster headache, and migraine.

3. The method of claim 1, wherein the individual has an insufficient therapeutic response or adverse effects after the use of other psychedelics substances and said method is used as a second-line treatment.

4. The method of claim 1, wherein the individual has a need for a qualitatively different psychedelic response after the use of other psychedelics substances and said method is used as an alternative treatment option.

5. The method of claim 1, wherein the individual has a need for a more attenuated response, with a slower onset of the psychological, or physiological response of the psychedelic (attenuated and prolonged response) compared with other psychedelics and said inducing step provides an effect chosen from the group consisting of less nausea and vomiting than psilocybin, less cardiovascular stimulation than psilocybin, reduced thermogenic acute effects compared with psilocybin, less bad drug effects including anxiety, fewer or less intensive headaches than psilocybin, an overall slow and attenuated effect onset compared with psilocybin, reduced peak response at longer effect duration and overall effect than comparable treatment options such as psilocybin, an overall intensive subjective experience while exhibiting a favorable acute adverse effects profile, and combinations thereof.

6. The method of claim 1, wherein said inducing step is performed in the individual to increase feelings of trust and openness beneficial in enhancing the therapeutic alliance and catalyze the effects of psychotherapy for any indication.

7. The method of claim 1, wherein said inducing step is performed in the individual to produce an inward oriented focus of attention and subjective insight to enhance psychotherapy.

8. The method of claim 1, wherein said inducing step is performed in the individual to induce neuroregenerative processes beneficial in medical conditions chosen from the group consisting of Alzheimer's disease, dementia, predementia, and Parkinson's disease.

9. The method of claim 1, wherein the composition is administered in a dose of 1-800 mg.

10. The method of claim 1, wherein said administering step is further defined as administering a dose chosen from the group consisting of a micro dose of mescaline hydrochloride (1-100 mg) inducing no to minimal subjective effects and equivalent to <20 μg of LSD base, a low dose of mescaline hydrochloride (100-200 mg) inducing mild psychedelic effects and equivalent to 20-40 μg of LSD, a moderate to medium dose of mescaline hydrochloride (300-400 mg) inducing a moderate to medium strong psychedelic experience with mainly positive drug effects and equivalent to 60-80 μg of LSD, a medium to high dose of mescaline hydrochloride (500 mg) equivalent to 100 μg of LSD base and inducing a full “good effect” psychedelic response with mainly positive drug effects and moderate ego-dissolution and a moderate risk of producing anxiety, and a high dose of mescaline hydrochloride (800 mg) equivalent to 150-200 μg of LSD base and inducing a full and very strong psychedelic response including marked “ego-dissolution” and having a high risk of producing anxiety.

11. A method of therapy, including the steps of:
administering an intermediate “good effect dose” of a composition chosen from the group consisting of mescaline, salt of mescaline, analogs thereof, and derivatives thereof to an individual; and
inducing positive acute drug effects that are known to be associated with more positive long-term responses in psychiatric patients.

12. The method of claim 11, wherein the “good effect dose” is further defined as 500 mg of the composition.

13. A method of therapy, including the steps of:
administering an “ego-dissolution” dose of a composition chosen from the group consisting of mescaline, a salt of mescaline, analogs thereof, and derivatives thereof to an individual; and
providing the experience of ego-dissolution.

14. The method of claim 13, wherein the “ego-dissolution” dose is further defined as 800 mg of the composition.
